(19) United States
(12) Patent Application Publication
(10) Pub. No.: US 2016/0358331 A1
(43) Pub. Date: Dec. 8, 2016

(54) REAL-TIME FOCUS FEEDBACK FOR MOBILE PHONE IMAGES USING BARCODE SYNCHRONIZATION ELEMENTS

(71) Applicant: Gaurav Sharma, Webster, NY (US)

(72) Inventor: Gaurav Sharma, Webster, NY (US)

(21) Appl. No.: 15/169,202

(22) Filed: May 31, 2016

Related U.S. Application Data
(60) Provisional application No. 62/169,681, filed on Jun. 2, 2015.

(51) Int. Cl. G06T 7/00; A61B 5/00; H04W 88/02; H04N 5/232; H04N 5/225

(52) U.S. Cl. CPC G06T 7/0012; H04N 5/23212; H04N 5/2257; H04W 88/02; A61B 5/0077; A61B 5/445; A61B 5/6898; G06T 2207/30204; G06T 2207/30088; G06T 2207/30196

(57) ABSTRACT

A method for autofocusing a camera of a mobile device while imaging a low contrast or less well-defined surface includes the steps of: providing a mobile device software application for image capture; affixing or printing at least one or more marker patterns on the low contrast or less well-defined surface; imaging by the mobile device camera the low contrast or less well-defined surface; identifying by the application the at least one or more marker patterns; focusing the mobile device camera by the application based on the at least one or more marker patterns; and acquiring an in focus image by the mobile device camera of the low contrast or less well-defined surface. An adhesive strip imaging assist device for autofocusing a mobile phone camera and an autofocus apparatus with marker pattern projector and marker pattern projector method are also described.

### CROSS-REFERENCE TO RELATED APPLICATIONS

This application claims priority to and the benefit of co-pending U.S. provisional patent application Ser. No. 62/169,681, REAL-TIME FOCUS FEEDBACK FOR MOBILE PHONE IMAGES USING BARCODE SYNCHRONIZATION ELEMENTS, filed Jun. 2, 2015, which application is incorporated herein by reference in its entirety.

### FIELD OF THE APPLICATION

The application relates to imaging low contrast or less well-defined surfaces and particularly to autofocus of a camera while imaging skin surfaces.

### BACKGROUND

Cameras are prevalent today throughout our lives. Smart phone and mobile tablet cameras are omnipresent and now have many uses beyond taking snapshot pictures. For example, some applications (apps) for smartphones and/or tablet computers can read barcodes, such as to provide a link to a website for manufacturer information, or to list product availability for comparison shopping.

### SUMMARY

According to one aspect, a method for autofocusing a camera of a mobile device while imaging a low contrast or less well-defined surface includes the steps of: providing a software application for the mobile device for image capture; affixing or printing at least one or more marker patterns on the low contrast or less well-defined surface where marker patterns have not been previously affixed; imaging by the mobile phone camera the low contrast or less well-defined surface; identifying by the application the at least one or more marker patterns; focusing the mobile device camera by the application based on the at least one or more marker patterns; and acquiring an in focus image by the mobile phone camera of the low contrast or less well-defined surface.

In one embodiment, the step of affixing or printing includes affixing or printing at least one or more marker patterns on the low contrast or less well-defined surface by a removable tattoo.

In another embodiment, the step of affixing or printing includes affixing or printing at least one or more marker patterns on the low contrast or less well-defined surface by at least one or more adhesive backed marker patterns.

In yet another embodiment, the step of affixing or printing includes affixing or printing at least one or more marker patterns on the low contrast or less well-defined surface by an adhesive bandage having at least one or more marker patterns printed thereon, and an open or transparent imaging window.

In yet another embodiment, the step of acquiring includes acquiring an in focus image by the mobile phone camera of the low contrast or less well-defined skin surface of a patient.

According to another aspect, an adhesive strip imaging assist device for autofocusing a mobile device camera while imaging a low contrast or less well-defined surface includes a strip substrate having a strip imaging surface and an adhesive backing on an opposite strip surface. An imaging window is disposed in the strip substrate to provide a view of the low contrast or less well-defined surface to be imaged. At least one or more marker patterns are disposed on the strip imaging surface about at a periphery of the imaging windowing.

In one embodiment, the strip substrate includes a bandage.

In another embodiment, the strip substrate includes a plastic.

In yet another embodiment, the strip substrate includes a fabric.

In yet another embodiment, the window includes an opening in the strip substrate.

In yet another embodiment, the window includes a substantially optically transparent window.

In yet another embodiment, the marker pattern includes a synchronization pattern of a barcode.

In yet another embodiment, the marker pattern includes a marker pattern of a QR barcode.

In yet another embodiment, the low contrast or less well-defined surface includes a skin surface of a patient.

According to yet another aspect, a method for autofocusing a camera of a mobile device while imaging a low contrast or less well-defined surface includes the steps of: providing a mobile device including a software application for image capture and a projection device to project a marker pattern onto a low contrast or less well-defined surface; projecting by the projecting device at least one or more marker patterns onto the low contrast or less well-defined surface; imaging by the mobile device camera the low contrast or less well-defined surface; identifying by the application the at least one or more marker patterns; focusing the mobile phone camera by the application based on the at least one or more marker patterns; and acquiring an in focus image by the mobile device camera of the low contrast or less well-defined surface.

According to yet another aspect, a mobile apparatus with a marker pattern based auto-focus assist system to improve a mobile device camera autofocus for imaging a less well-defined surface includes an imager and a processor to run an image capture software application. A projection device projects a marker pattern as a focusing target of a camera auto-focus system onto a less well-defined surface. The projection device is operated by the processor or another processor programmed to project the marker pattern onto the less well-defined surface.

In one embodiment, the projection device includes a LED or laser scanning type projection device.

The foregoing and other aspects, features, and advantages of the application will become more apparent from the following description and from the claims.

### BRIEF DESCRIPTION OF THE DRAWINGS

The features of the application can be better understood with reference to the drawings described below, and the claims. The drawings are not necessarily to scale, emphasis instead generally being placed upon illustrating the principles described herein. In the drawings, like numerals are used to indicate like parts throughout the various views.

FIG. 1 shows an exemplary QR barcode of the prior art having three marker patterns;

FIG. 2 shows an image of an exemplary surface of a patient's skin having dermatological features;

FIG. 3 is an illustration showing three exemplary marker patterns as contemplated marked or printed on a skin surface;

FIG. 4 is an illustration of an exemplary bandage with marker patterns and a window to reveal the skin surface;

FIG. 5 is a diagram showing an exemplary method for autofocusing a camera of a mobile device while imaging a low contrast or less well-defined surface

FIG. 6 is a drawing showing an exemplary mobile apparatus with an imager and marker pattern projector device; and

FIG. 7 is a block diagrams showing the mobile device camera, and projection device of the exemplary mobile apparatus of FIG. 6.

### DETAILED DESCRIPTION

### Definitions

Less well defined surface, low contrast surface: A less well defined surface or a low contrast is taken herein to include surfaces having low contrast between light and dark regions. Such surfaces typically have few if any well-defined relatively high contrast lines or edges. Where well-defined relatively high contrast features are present, such features generally occupy a small percentage of the overall surface to be imaged, typically less than about 25% of the surface area.

Marker patterns: Marker patterns include standardized patterns that are used for synchronization of barcodes. As contemplated herein, marker patterns typically occupy a small percentage of the surface area to be imaged (e.g. less than about 25%). However, unlike relatively high contrast marks which may be happenstance present on a less well defined surface to be imaged, a process algorithm, such as, for example, portions of barcode reading applications, can identify one or more marker patterns and autofocus specifically on the surface areas occupied by the one or more marker patterns of the less well-defined surface.

Mobile device: includes any cell phone (including phones wired or wirelessly connected by any suitable means, such as for example, WiFi connected phones), smart phones running any suitable operating system (e.g. Android or Apple iOS), tablets, iPads, etc. As used herein, a smart phone is an example of a mobile device. It will be appreciated by those skilled in the art that the focus techniques described hereinbelow can also apply to tablets, iPads, notebook computers, netbook computers, portable computers, etc. and any other suitable portable computing devices with a processor and a camera, without regard to the presence of an integral cell phone function.

Application: Application as used herein (other than use in the context to the patent application), refers to a computer executable code typically running on the mobile device or on a processor or processor equivalent of any other suitable portable computing device. Mobile device applications are also commonly referred to as “apps”.

As described hereinabove, smartphone cameras are being used for a wide variety of applications beyond sharing snapshot pictures with friends and family For example, patients have been able to take pictures of various skin conditions in various states of inflammation or distress to show to show a skin condition to a dermatologist by email, or during an office visit (e.g. to show a skin condition not present at the time of the visit).

Most modern smartphone cameras include active focus control systems which can autofocus under smartphone/camera firmware and/or software control. However, if there is insufficient definition on the surface being imaged, most autofocus systems perform poorly, or not at all. Photographs of skin are a particularly good example of a surface where prior art smartphone camera autofocus systems have trouble performing an efficient or successful autofocus operation. Even where there is some skin pathology, such as a reddened swollen area, there still may be little definition on the skin surface to help an autofocus system set the correct focus under programmatic control.

Adding edges or lines, such as by writing or printing on the surface of the skin with a surgical pen marker can help, however such markings can inadvertently mask the very pathology the doctor should be viewing to make a diagnosis or to review progress or healing from an ailment. While small marks at the periphery of the surface to be imaged by a smartphone camera can be helpful, most prior art autofocus systems focus across the entire imaging area. Unfortunately, for such autofocusing strategies, small marks at the periphery that occupy, for example about 5% to 10% of the surface area, or even less than about 25%, are insufficient for reliable camera autofocus operation. Also, such line and edge enhancements to help an off the shelf smart phone to focus will have varying and likely inconsistent results across different models and manufacturer's phone cameras and autofocus routines Improper focus can be especially problematic for images captured using mobile phones.

When imaging barcodes, the synchronization elements from common 2-D barcodes such as the QR codes provide real-time feedback for autofocusing systems which are typically controlled by a barcode reading application. Synchronization elements, such as the finder patterns or marker patterns in QR codes, are designed to be rapidly identifiable and locatable in a well-focused image allowing real-time localization of the barcode when it is included in the field of view.

It was realized that beyond use of marker patterns in barcodes, the marker patterns of barcodes themselves can provide a solution to the problem of camera autofocus where a surface to be imaged is less well defined (e.g. low contrast with few if any light/dark sharp boundaries). In other words, barcode marker patterns (in the absence of other parts of the barcode such as the data elements), can solve the problem of the inability of camera autofocus systems to focus on low definition surfaces.

A solution to the problem of autofocus of a less well defined surface is to employ synchronization patterns (marker patterns) from 2-D barcodes, such as on or in a target attached to the object being imaged. Such marker patterns can be included on a surface by any suitable means, such as for example, by printing on the surface, by taping one or more marker patterns on the surface, or by an adhesive backed surface substrate with one or markers, such as for example, an adhesive backed surface substrate with one or markers with an open or transparent imaging window. By so using barcode like marker patterns image, by including these marker pattern elements in the field of view, image capture can be performed with improved autofocus operations, and improved image focus.

Real-time Focus Feedback for Mobile Phone Images Using Barcode Synchronization Elements: Focus problems are a frequent source of problems in images captured using mobile phones. Because the preview on the phone is quite small in size, these focus problems are very hard to diagnose using the image shown on the phone display, and can still pose problems when the image is sent to a larger display or used for zooming in to see specific detail. As a solution, detection of the synchronization patterns can allow a suitably designed mobile phone application for the image capture to computationally identify when the image is in focus. Such use of a mobile phone application can substantially eliminate the capture of poorly focused images and also provide the user real-time feedback on any focus problems.

The autofocus techniques based on the addition of one or more marker patterns on a surface to be imaged as described herein are applicable in a wide variety of situations for imaging a low contrast or less well-defined surface where focus errors would otherwise typically occur while capturing images with mobile phones. One exemplary application for the new technique and autofocus assist devices is for dermatology imaging. For example, in medical applications, such techniques are contemplated to be useful, for example, for imaging an area of a patient's skin surface.

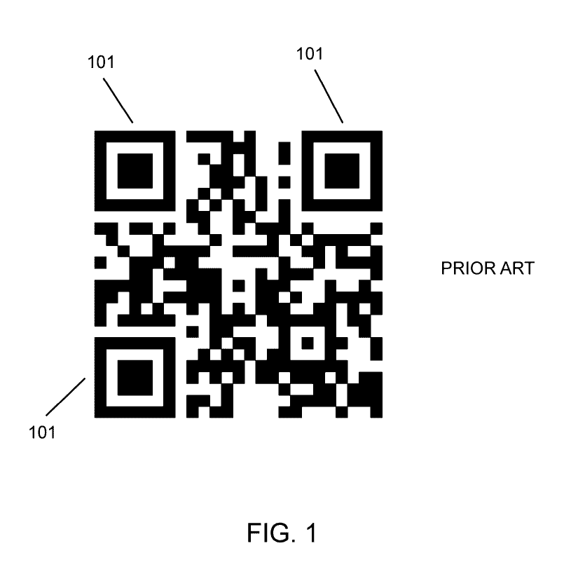

FIG. 1 shows a QR barcode of the prior art having three marker patterns 101. Barcode marker patterns 101 are portions of a barcode dedicated to helping the barcode reader find, register, and to autofocus on the barcode before decoding the data embedded in the barcode. 2-D barcodes include marker blocks which are also typically referred to as synchronization blocks, finder patterns, or pilot blocks. Such features and associated barcode reading techniques have been described, for example, in issued U.S. Pat. No. 9,111,186, COLOR BARCODES FOR MOBILE APPLICATIONS: A PER CHANNEL FRAMEWORK, and in issued U.S. Pat. No. 8,047,447, METHOD FOR ENCODING AND DECODING DATA IN A COLOR BARCODE PATTERN, and U.S. Pat. No. 8,100,330, METHOD FOR ENCODING AND DECODING DATA IN A COLOR BARCODE PATTERN, and in ISO/IEC 18004:2006 Information technology—Automatic identification and data capture techniques—QR code 2005 bar code symbology specification, published Sep. 1, 2006. Both of the '685 application and the '447, '330 patents are incorporated herein by reference in their entirety for all purposes.

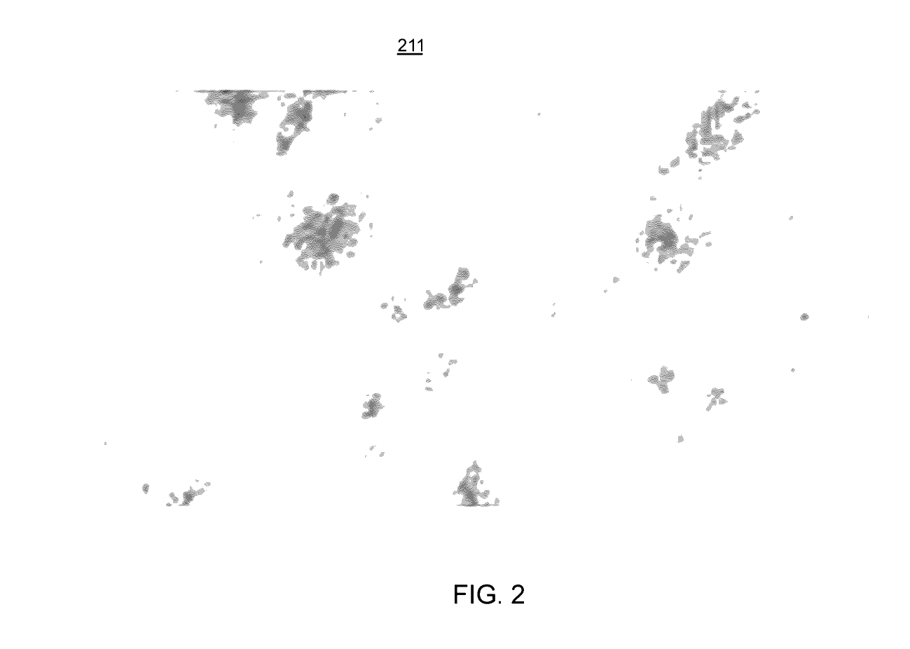

FIG. 2 shows an image of an exemplary surface of skin having dermatological features. An autofocus camera, such a mobile phone camera can have difficulty focusing on such a surface because of the lack of well-defined high contrast features. As described hereinabove, merely adding parts of a barcode around the periphery of an ill-defined surface (e.g. a less well-defined skin surface) to be imaged may not fill a high enough percentage of the surface to be imaged to provide a reliable repeatable accurate autofocus operation.

However, by using a barcode decoding process, a processor of a mobile phone can search for one or more marker patterns 101 in an image. Once a suspected marker pattern 101 is identified, the process can cause the camera's autofocus operation to adjust to make the one or more marker patterns 101 readable by the application. That is, for the autofocus operation, the camera considers one or more marker patterns to be the areas of the surface to be used for the autofocusing operation. As the marker patterns come into focus and the application can properly read or otherwise process a marker pattern 101, the camera has properly focused on the surface on which the one or more markers reside. Thus the camera can take an in focus image of the low contrast or less well-defined surface (e.g. a surface of skin of a patient). In such situations of imaging the low contrast or less well-defined surface, the normal autofocus function of the camera would otherwise have likely resulted in an out of focus image.

In some embodiments, it is contemplated that three or more marker patterns 101 are placed around about the periphery of a surface to be imaged. The three or more marker patterns 101 can help to ensure that substantially the entire surface to be imaged is in focus when all of the three or more marker patterns can be successfully read by the marker pattern decoding and autofocus process.

Such marker patterns can be printed onto the about the periphery of the surface by any suitable printing means. For example, three or more marker patterns can be added by about the periphery of a skin surface to be imaged by any suitable tattoo transfer method, such as, for example by temporary tattoos in common use today for body art applications. One advantage of a temporary tattoo is that it can be applied to a patient's skin so as to last for several days. With the static nature of a marker printed on the skin, the marker patterns, in addition to providing the autofocus assist function, the marker patterns can also provide a static geographic reference on a surface area of skin.

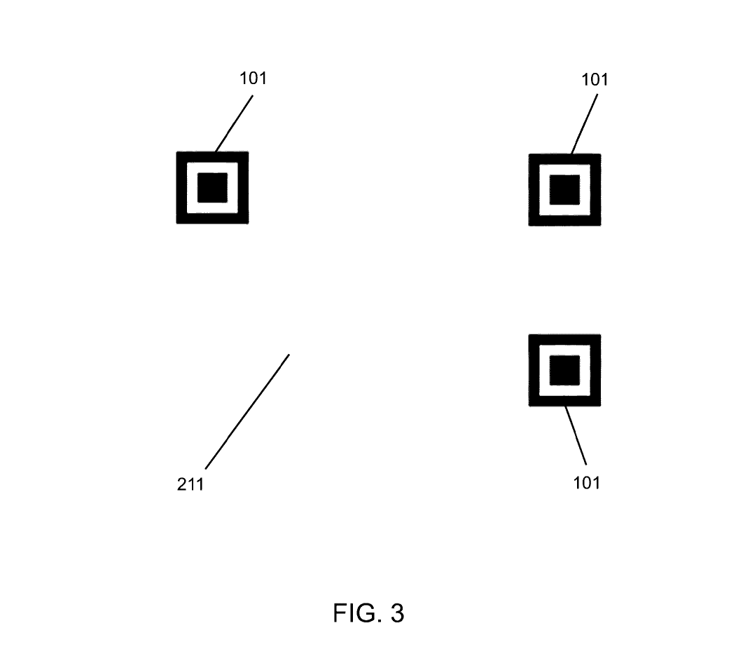

FIG. 3 is an illustration showing three marker patterns 101 marked as contemplated on a skin surface such as the skin surface of FIG. 2. Marker patterns 101 such as are shown in FIG. 3 can be applied, for example, by one or more removable tattoo sheets. Such removable tattoo sheets application techniques and devices (e.g. ink transfer methods and sheets) are well known to those skilled in the art.

In other embodiments, an adhesive strip autofocus imaging assist device can be made from a strip substrate having a strip imaging surface. The strip autofocus imaging assist device can also have an adhesive backing on an opposite strip surface, such as, for example, a section of tape or any suitable substrate with an adhesive backing. The strip autofocus imaging assist device can have an open window or an optically transparent window for unobstructed imaging of an area of skin, while at the same time, providing a substrate on which to print one or more marker patterns on the imaging surface around about the periphery of the window.

The strip autofocus imaging assist device can be manufactured, for example, as a bandage which can be made from any suitable material such as plastics (e.g. a pliable plastic) or a fabric as known in the art. In some embodiments, the strip autofocus imaging assist device can be similar to an adhesive bandage (e.g. a BANDAID™) with a window cut in what would otherwise be a traditional padded area of the bandage intended to cover an open wound. Such applications of marker patterns by bandage are contemplated to be relatively comfortable for a patient to wear during a diagnostic imaging period ranging, for example, from hours to days.

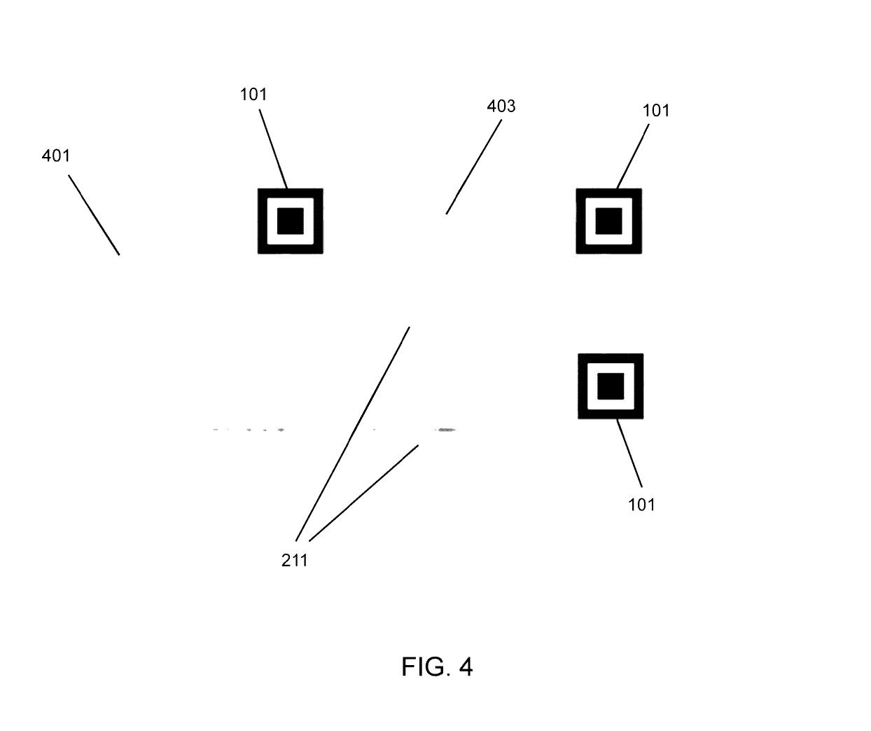

FIG. 4 is an illustration showing an exemplary contemplated bandage 401 with marker patterns 101 and a window 403 to reveal the skin surface 211. Marker patterns 101 are disposed around window 403 and printed on the surface of the bandage 401. It is unimportant whether the light areas of the marker pattern are white as shown in FIG. 4, or a color of the strip imaging surface of bandage 401.

Bandage 401 can have any suitable means of affixing it to the surface of the skin surface 211 for various environmental conditions (rain, bathing, etc.) and for various intended wearing time (e.g. for one image, for a few days, or for a week or more). Suitable adhesives (not shown in FIG. 4) for such bandage applications are well known in the art.

A process algorithm, typically as an application or as a portion of an application, can run on a smartphone which controls the camera. The process algorithm searches the image for one or more marker patterns using techniques as are presently used to read a barcode (e.g. to read a QR barcode). Once one or more marker patterns are found on the surface, the process algorithm causes the camera to focus on the one or more marker patterns independent of what percentage of the frame is filled by the one or more marker patterns. Once the marker pattern is in focus, the camera can take one or more pictures of the surface of the skin using the focus setting determined by autofocusing on the one or more marker patterns.

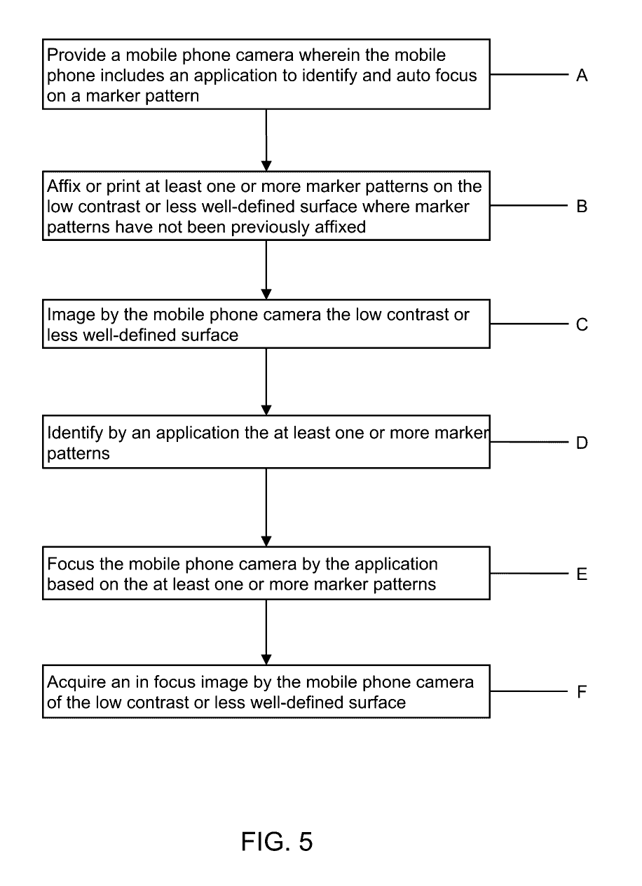

FIG. 5 shows an exemplary method for autofocusing a mobile phone camera of a mobile phone while imaging a low contrast or less well-defined surface including the steps of A) Provide a mobile phone camera wherein the mobile phone includes an application to identify and autofocus on a marker pattern; B) Affix or print at least one or more marker patterns on the low contrast or less well-defined surface (or make use of such patterns previously affixed); C) Image by the mobile phone camera the low contrast or less well-defined surface; D) Identify by an application the at least one or more marker patterns; E) focus the mobile phone camera by the application based on the at least one or more marker patterns; and F) Acquire an in focus image by the mobile phone camera of the low contrast or less well-defined surface.

As described hereinabove, marker patterns can be included on a surface by any suitable means, such as for example, by printing on the surface, by taping one or more marker patterns on the surface, or by an adhesive backed surface substrate with one or markers, such as for example, an adhesive backed surface substrate with one or markers with an open or transparent imaging window. It is also contemplated that such marker can be projected onto the surface. For example, it is contemplated that a camera can include a projection device that allows for projection of any suitable “finder patterns” onto a surface being imaged. The camera can then adjust focus based on the detection and analysis of the projected pattern in the image. The projection device can use any suitable illumination projection means, including, for example, LED or laser projection means. Typically, such projection means including scanning means such as, for example, scanning mirrors to project a 2D image on the surface.

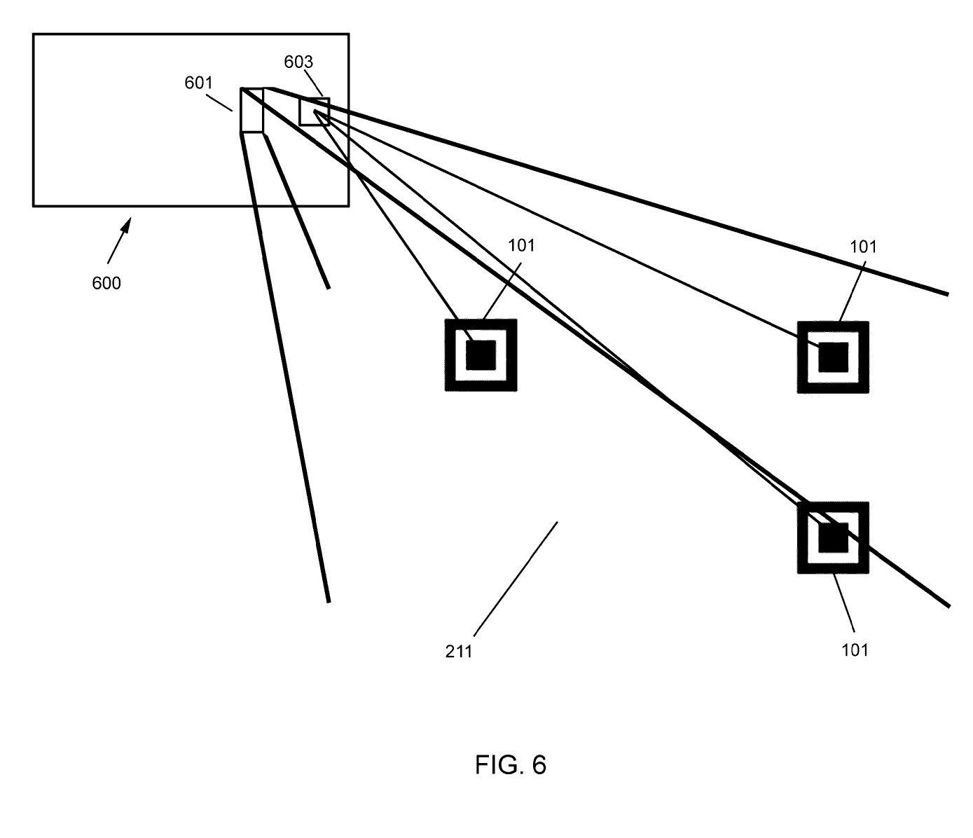

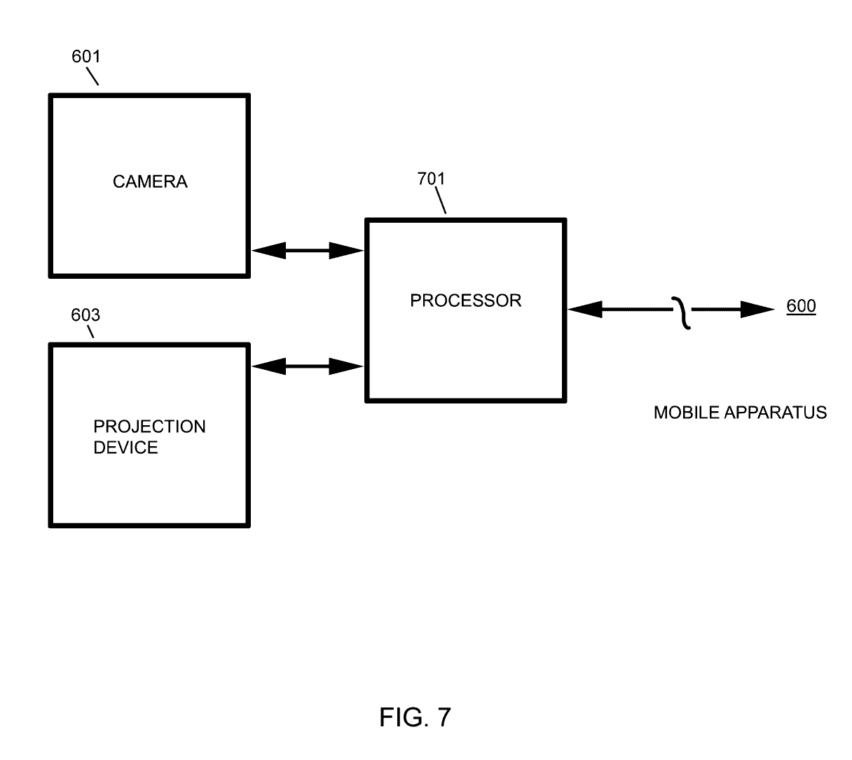

FIG. 6 is a drawing showing an exemplary mobile apparatus 600 with an imager and marker pattern projector. The mobile apparatus 600 includes an imager 601 and a processor to run an image capture software application. A projection device 603 projects a marker pattern 101 as a focusing target of a camera auto-focus system onto a less well-defined surface 211. The projection device is operated by a processor (701, FIG. 7) or another processor programmed to project said marker pattern onto said less well-defined surface.

FIG. 7 is a block diagrams showing the mobile device camera, and projection device of the exemplary mobile apparatus of FIG. 6.

It is understood that the techniques described herein with respect to mobile phone cameras can also apply to any mobile computing device as currently known or may be later introduced that has an internal or an external camera, with a focus control or adjustment which can be programmatically by internal or external firmware or software.

The process algorithm is typically downloaded, as in downloading an app, to a computer readable non-transitory storage medium, such as a computer readable non-transitory storage medium of a smartphone. A computer readable non-transitory storage medium as non-transitory data storage includes any data stored on any suitable media in a non-fleeting manner. Such data storage includes any suitable computer readable non-transitory storage medium, including, but not limited to hard drives, non-volatile RAM or EEPROM or any other suitable type of mobile phone non-volatile memory, such as a smart phone), SSD devices, CDs, DVDs, etc.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A method for autofocusing a camera of a mobile device while imaging a low contrast or less well-defined surface comprising the steps of:
providing a software application for the mobile device for image capture;
affixing or printing at least one or more marker patterns on a low contrast or less well-defined surface;
imaging by said mobile device camera the low contrast or less well-defined surface;
identifying by the application said at least one or more marker patterns;
focusing said mobile phone camera by said application based on said at least one or more marker patterns; and
acquiring an in focus image by said mobile device camera of said low contrast or less well-defined surface.

2. The method of claim 1, wherein said step of affixing or printing comprises affixing or printing at least one or more marker patterns on said low contrast or less well-defined surface by a removable tattoo.

3. The method of claim 1, wherein said step of affixing or printing comprises affixing or printing at least one or more marker patterns on said low contrast or less well-defined surface by at least one or more adhesive backed marker patterns.

4. The method of claim 1, wherein said step of affixing or printing comprises affixing or printing at least one or more marker patterns on said low contrast or less well-defined surface by an adhesive bandage having at least one or more marker patterns printed thereon, and an open or transparent imaging window.

5. The method of claim 1, wherein said step of acquiring comprises acquiring an in focus image by said mobile device camera of said low contrast or less well-defined skin surface of a patient.

6. An adhesive strip imaging assist device for autofocusing a mobile device camera while imaging a low contrast or less well-defined surface comprising:
a strip substrate having a strip imaging surface and an adhesive backing on an opposite strip surface;
an imaging window disposed in said strip substrate to provide a view of the low contrast or less well-defined surface to be imaged; and
at least one or more marker patterns disposed on said strip imaging surface about at a periphery of said imaging windowing.

7. The adhesive strip imaging assist device of claim 6, wherein said strip substrate comprises a bandage.

8. The adhesive strip imaging assist device of claim 6, wherein said strip substrate comprises a plastic.

9. The adhesive strip imaging assist device of claim 6, wherein said strip substrate comprises a fabric.

10. The adhesive strip imaging assist device of claim 6, wherein said window comprises an opening in said strip substrate.

11. The adhesive strip imaging assist device of claim 6, wherein said window comprises a substantially optically transparent window.

12. The adhesive strip imaging assist device of claim 6, wherein said marker pattern comprises a synchronization pattern of a barcode.

13. The adhesive strip imaging assist device of claim 6, wherein said marker pattern comprises a marker pattern of a QR barcode.

14. The adhesive strip imaging assist device of claim 6, wherein the low contrast or less well-defined surface comprises a skin surface of a patient.

15. A method for autofocusing a camera of a mobile device while imaging a low contrast or less well-defined surface comprising the steps of:
providing a mobile device comprising a software application for image capture, and a projection device to project a marker pattern onto a low contrast or less well-defined surface;
projecting by said projecting device at least one or more marker patterns onto said low contrast or less well-defined surface;
imaging by said mobile device camera the low contrast or less well-defined surface;
identifying by the application said at least one or more marker patterns;
focusing said mobile phone camera by said application based on said at least one or more marker patterns; and
acquiring an in focus image by said mobile device camera of said low contrast or less well-defined surface.

16. A mobile apparatus with a marker pattern based auto-focus assist system to improve a mobile device camera autofocus for imaging a less well-defined surface comprising:
an imager and a processor to run an image capture software application; and
a projection device to project a marker pattern as a focusing target of a camera auto-focus system onto a less well-defined surface, said projection device operated by said processor or another processor programmed to project said marker pattern onto said less well-defined surface.

17. The mobile apparatus of claim 16, wherein said projection device comprises a LED or laser scanning type projection device.
